(19) United States
(12) Patent Application Publication
(10) Pub. No.: US 2016/0339186 A1
(43) Pub. Date: Nov. 24, 2016

(54) SKIN ADHESIVE DEVICE FOR USE IN MEDICAL PROCEDURES

(71) Applicant: SOMA ACCESS SYSTEMS, LLC, Englewood, CO (US)

(72) Inventors: M. Dexter Hagy, Greenville, SC (US); Stephen F. Ridley, Greenville, SC (US)

(21) Appl. No.: 15/106,559

(22) PCT Filed: Dec. 17, 2014
(86) PCT No.: PCT/US14/70885

§ 371 Date: Jun. 20, 2016

Related U.S. Application Data
(60) Provisional application No. 61/919,109, filed on Dec. 20, 2013.

(51) Int. Cl. A61M 5/42; A61B 90/00

(52) U.S. Cl. CPC A61M 5/425; A61B 90/02; A61B 2017/00951

(57) ABSTRACT

A skin adhesive device is described that can be used in conjunction with a variety of medical devices that are held against a skin surface and/or that puncture the skin during a medical procedure. The device can be adhered to the skin and can hold the skin taut during a procedure so as to maintain the skin against a medical device and/or to prevent compression of subdermal structures during a procedure. Following use, the device can be simply removed from the skin surface and discarded.

### CROSS REFERENCE TO RELATED APPLICATION

This application claims filing benefit of U.S. Provisional Patent Application Ser. No. 61/919,109 having a filing date of Dec. 20, 2013, which is incorporated herein by reference.

### BACKGROUND

A variety of medical procedures include holding or pressing a medical device against the skin. For instance, ultrasound guided procedures utilize an ultrasound device held against the skin surface to visualize a subdermal target such as a blood vessel and thereby improve guidance of a probe, a needle, or the like to the subdermal target. Other devices can be held against the skin for other medical procedures such as for guidance purposes in access of fistulas during dialysis, for insertion of materials or implants, or for removal of fluids, masses or implants from a patient.

Unfortunately, it is often difficult to hold a medical device tightly against the skin during a procedure. For instance, when utilizing an ultrasound device, it is often beneficial to angle the device at the skin surface in order to obtain a wider field of view or to get a better view of a target, e.g., a blood vessel, tumor, nerve bundle, etc. Unfortunately, when the device is held at an angle to the skin, an air gap can form between the transducer surface of the device and the skin, which can detrimentally affect the visualization procedure. Relative motion between the skin and the medical device can also be a problem when inserting or removing material from a patient. Similarly, other procedures such as puncturing the skin with a needle for IV access, compresses the very structure that is targeted.

What is needed in the art is a device and method that can hold the skin surface against the surface of a medical device during a medical procedure and/or lift the skin during a skin puncture procedure as to prevent compression of subdermal structures.

### SUMMARY

Aspects and advantages of the invention will be set forth in part in the following description, or may be obvious from the description, or may be learned through practice of the invention.

According to one embodiment, disclosed is a skin adhesive device. The device can include a gripping tab and a skin contacting member. The gripping tab can include a proximal end and a distal end and a first side and a second side. The skin contacting member includes a top side and a bottom side and has a length and a width. The gripping tab extends from the top side of the skin contacting member with the proximal end of the gripping tab nearer the top side of the skin contacting member as compared to the distal end of the gripping tab. The bottom side of the skin contacting member includes a biocompatible adhesive.

Also disclosed is a method for utilizing the skin adhesive device. For instance, the method can include adhering the biocompatible adhesive on the bottom side of the skin contacting member to a skin surface and maintaining a pulling pressure on the gripping tab during the medical procedure. The pulling pressure can pull the skin that is adhered to the adhesive and can maintain the skin taut during the medical procedure.

In one embodiment, the skin adhesive device can be utilized in conjunction with a medical device. For instance, a method of using the skin adhesive device can also include placing one surface of a medical device adjacent to at least a portion of the top surface of the skin contacting member of the skin adhesive device and placing another surface of the medical device against one side of the gripping tab. The gripping tab can then be held against the medical device as the medical device is utilized to carry out a procedure.

When the gripping tab is held, for instance in conjunction with a medical device, and the medical device is moved or rocked at the skin surface, the skin contacting member that is adhered to the skin will pull and stretch the skin so that the skin and the medical device surface can remain adjacent to one another and so that there is little or no relative motion between the skin and the medical device held against the skin. The adhesive device can stretch the skin and keep it taut against the medical device throughout the procedure.

### BRIEF DESCRIPTION OF THE FIGURES

A full and enabling disclosure of the present invention, including the best mode thereof, directed to one of ordinary skill in the art, is set forth in the specification, which makes reference to the appended figures, in which:

FIG. 1 includes FIG. 1A illustrating one embodiment of a skin adhesive device and FIG. 1B illustrating one embodiment of a skin contacting member of a device.

FIG. 2 illustrates an exploded view of the skin adhesive device of FIG. 1.

FIG. 3 includes FIG. 3A and FIG. 3B that illustrate one method for forming a device.

FIG. 4 includes FIG. 4A and FIG. 4B illustrating other methods for forming a device.

FIG. 5A illustrates a top view of the skin adhesive device of FIG. 1.

FIG. 5B illustrates a side view of the skin adhesive device of FIG. 1.

FIG. 5C illustrates an end view of the skin adhesive device of FIG. 1.

FIG. 6A illustrates another embodiment of a skin adhesive device.

FIG. 6B illustrates a top view of the skin adhesive device of FIG. 6A.

FIG. 6C illustrates a side view of the skin adhesive device of FIG. 6A.

FIG. 6D illustrates an end view of the skin adhesive device of FIG. 6A.

FIG. 7 illustrates an exploded view of the skin adhesive device of FIG. 6.

FIG. 8 illustrates one use for the skin adhesive device disclosed herein.

FIG. 9 presents a top view, side view, and perspective view of one embodiment of a skin adhesive device.

FIG. 10 presents a top view, side view, and perspective view of one embodiment of a skin adhesive device.

FIG. 11 presents a top view, side view, and perspective view of one embodiment of a skin adhesive device.

FIG. 12 presents a top view, side view, and perspective view of one embodiment of a skin adhesive device.

FIG. 13 presents a top view, side view, and perspective view of one embodiment of a skin adhesive device.

FIG. 14 presents a top view, side view, and perspective view of one embodiment of a skin adhesive device.

FIG. 15 presents a top view, side view, and perspective view of one embodiment of a skin adhesive device.

### DETAILED DESCRIPTION

It is to be understood by one of ordinary skill in the art that the present discussion is a description of exemplary embodiments only, and is not intended as limiting the broader aspects of the present disclosure. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

In general, disclosed herein is a skin adhesive device that can be used to hold an area of skin taut, for instance in conjunction with a variety of medical devices that are held against a skin surface and/or that puncture the skin during a medical procedure. For instance, the device can be adhered to the skin and can hold the skin taut during insertion of a subdermal device, such as a needle through the taut skin without compression of the subdermal architecture beneath the skin during the insertion. In one embodiment, the skin adhesive device can be held in conjunction with a medical device so as to stretch the skin as necessary to prevent compression of subdermal structures and keep the skin adjacent to a surface of the device during a procedure. Following use, the device can be simply removed Thorn the skin surface and discarded.

The ability to hold a medical device tightly against the skin surface can prevent the formation of an air gap between the medical device and the skin and can also prevent relative motion between the skin and the medical device. This can be beneficial in a variety of applications including, for example, diagnostic applications (e.g., visualization or detection of subdermal structures or materials), delivery applications (e.g., delivery of a medicine and/or an implantable device), or removal applications (e.g., retrieval of an implanted device or removal of a fluid or tissue mass). The ability to apply a lifting force to the skin can also oppose needle puncture force during a skin puncture procedure and can prevent compression and displacement of subdermal structures. This is beneficial in any needle access procedure where it is desirable to cannulate a target structure without transfixing the entire structure, for example, vascular access, such as during a dialysis process, when the goal is to puncture the anterior wall of vessel while avoiding puncturing the posterior wall of the vessel.

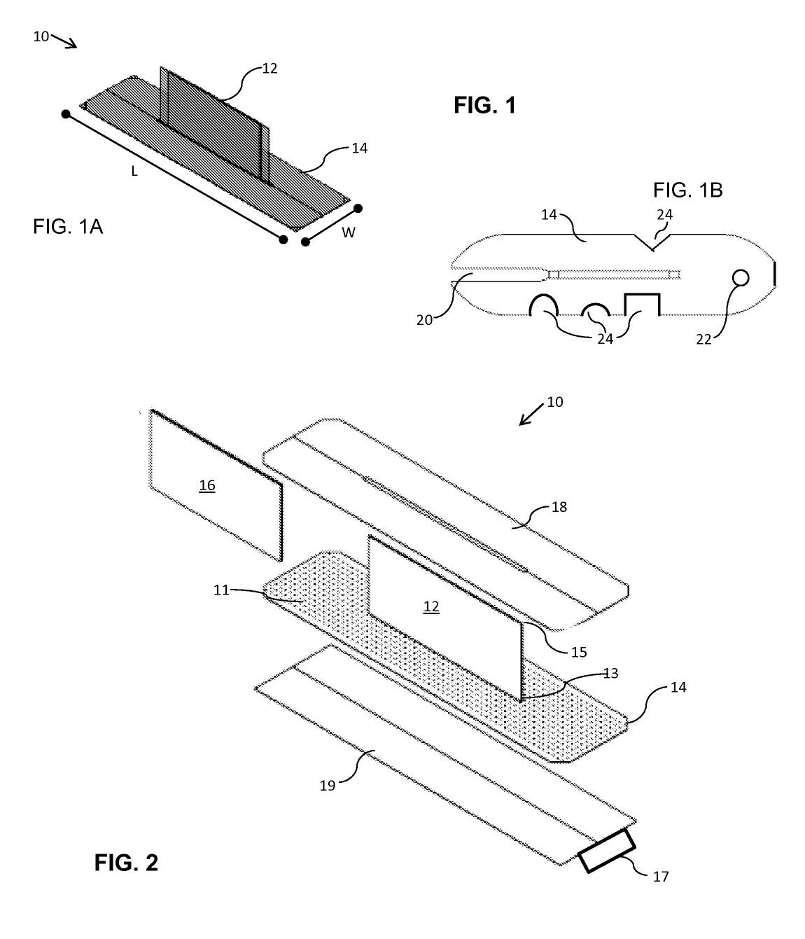

FIG. 1A illustrates one embodiment of the device 10. The device 10 includes a gripping tab 12 and a skin contacting member 14. The device 10 has a length L and a width W that in this particular embodiment are defined by the length and width of the skin contacting member 14. The length L of the device 10 can generally be from about ½ inch (about 1.3 centimeters) to about 12 inches (about 30.5 centimeters), or from about 1 inch (about 2.5 centimeters) to about 10 inches (25 centimeters) in some embodiments, or from about 1.5 inches (about 3.8 centimeters) to about 8 inches (about 20 centimeters) in some embodiments, or from about 2 inches (about 5 centimeters) to about 8 inches (about 20 centimeters) in some embodiments.

The width of the device 10 can generally be from about ½ inch (about 1.3 centimeters) to about 6 inches (about 15 centimeters), or from about 0.75 inches (about 2 centimeters) to about 4 inches (about 10 centimeters) in some embodiments, or from about 1 inch (about 2.5 centimeters) to about 3 inches (about 7.5 centimeters) in some embodiments, or from about 1.5 inches (about 3.8 centimeters) to about 2.5 inches (about 6.4 centimeters) in some embodiments.

FIG. 2 presents an exploded view of the device 10. As can be seen, the gripping tab 12 meets the top surface 11 of the skin contacting member 14 and extends away from the skin contacting member 14 from a proximal end 13 to a distal end 15.

The gripping tab 12 and the skin contacting member 14 can be formed of the same or different materials. For instance, the gripping tab 12 and/or the skin contacting member 14 can be formed of a fibrous material such as a woven, knitted, or nonwoven fabric formed of fibers that can be held adjacent to the skin. In one embodiment, the gripping tab 12 and/or the skin contacting member 14 can be formed of a polymeric material that can include a polymer in conjunction with additives as are generally known in the art including, for instance, colorants, nucleation agents, fillers (e.g., fibrous fillers), and so forth. For instance, the gripping tab 12 and/or the skin contacting member 14 can be a flexible or inflexible polymer-based material that can be formed according to any suitable formation process including, and without limitation, extrusion, injection molding, hot stamping, thermoforming, solution or melt casting, and so forth.

For instance, a flexible skin contacting member 14 exhibit stretch in one or more directions and/or can be pliable and an inflexible skin contacting member 14 can generally refer to a structure that can remain horizontal when supported by a single edge or end in a horizontal plane, e.g., it will not fold or bend without added pressure. However, in some embodiments, an inflexible skin contacting member may be shaped, e.g., bent, upon application of pressure while in other embodiments, an inflexible skin contacting member may be brittle and may not bend to any great extent, but rather may break or crack upon application of a bending force.

The gripping tab 12 and the skin contacting member 14 may be formed from natural, synthetic, or naturally occurring materials that can be synthetically modified in some embodiments, such as polysaccharides (e.g., cellulose materials such as paper and cellulose derivatives, such as cellulose acetate and nitrocellulose); polyether sulfone; nylon; polyvinylidene fluoride (PVDF); polyester; silica; cloth, both naturally occurring (e.g., cotton) and synthetic (e.g., nylon or rayon); polymeric materials, such as polyacrylamide, polyolefins (polyethylene, polypropylene, etc.), and so forth.

The skin contacting member 14 may be of any flexible or inflexible material suitable for being placed in contact with skin. In one embodiment, a flexible skin contacting member 14 can be made from an elastomer of synthetic rubbery material, such as an amorphous (when unstretched) synthetic polymer of relatively high molecular weight, for instance in the range from about 10,000 to about 1,000,000. Such a polymer can rapidly return to approximately its initial dimensions and shape after substantial deformation by a weak stress and release of the stress. Elastomers particularly useful can be elastic at room temperature, that is, they are above their glass transition temperature (T_{g}).

Examples of suitable elastomers include, without limitation, ethylene-co-vinyl acetate; ethylene-co-methylacrylate; butadiene-co-acrylonitrile; isobutylene-co-isoprene; terpolymer of ethylene, propylene and a diene sidechain; ethyl acrylate-co-butadiene; Estane™ brand urethanes formed by the reaction of (a) diisocyanates and polyalkylene polyether glycols, or (b) diisocyanates and polyalkylene polyester glycols; Hycar* brand butadiene-acrylonitrile modified with carboxyl groups; Hydrin™ brand copolymer of epichlorohydrin with ethylene oxide; styrene-butadiene-styrene block copolymers available under the Kraton™ brand; Hytrel™ brand copolyester, and the like, which are essentially liquid-impervious, do not absorb a significant amount of moisture, and can be formed into thin smooth surfaced sheets in the range from about 0.5 mil to about 10 mil thick, for instance from about 1 mil to about 5 mil in some embodiments.

An inflexible skin contacting member can be formed of a polymeric material, such as a polyolefin, a polycarbonate, a polyurethane, and so forth, that can be molded according to any suitable process and formed to the desired shape.

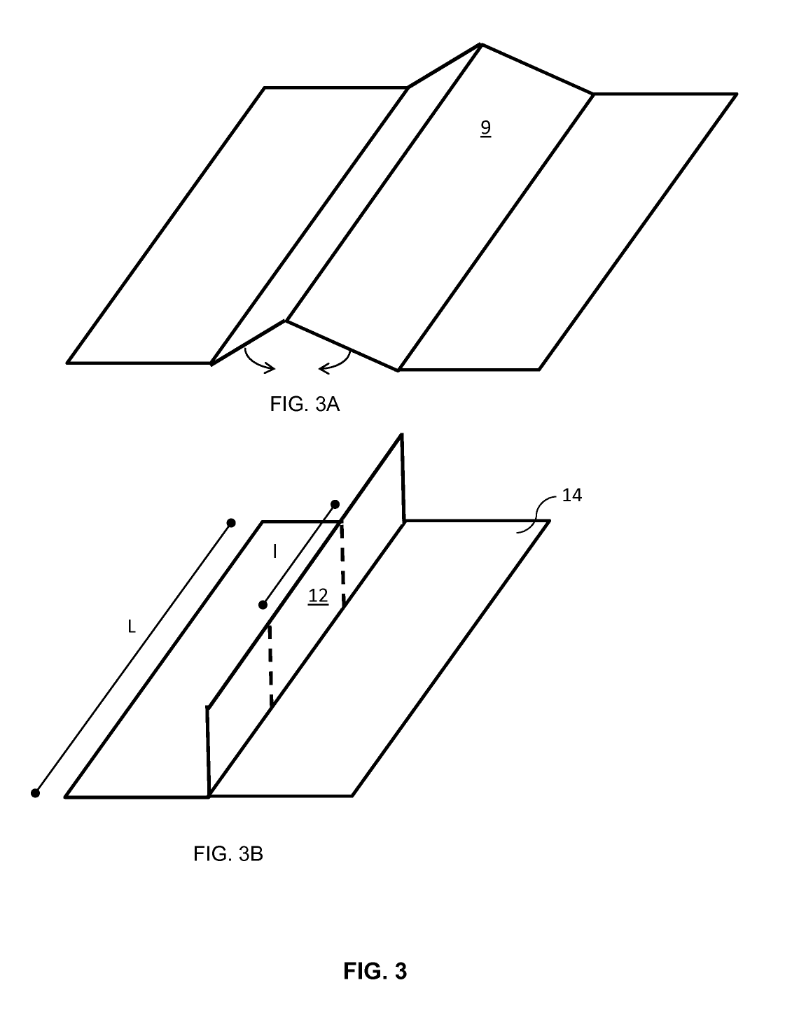

In one embodiment, the gripping tab 12 and skin contacting member 14 can be formed of a single piece of flexible or inflexible material that has been folded and optionally trimmed to form the two elements of the device. A formation method for this embodiment is illustrated in FIG. 3A, which includes a single sheet, film or other type of flexible or inflexible member 9 folded such that a center portion of the member 9 forms an inverted V shape. The inner sides of this shape can then be pulled together as illustrated by the directional arrows in FIG. 3A and adhered to one another to form the three dimensional shape at FIG. 3B that includes the skin contacting member 14 and the gripping tab 12 that extends from the skin contacting member 14. In one embodiment, the gripping tab can be trimmed as designated by the dashed lines such that the length L is greater than the length of the gripping tab 12 l, but this is not a requirement, and in other embodiments, the length l of the gripping tab 12 can be the same as or even greater than the length L of the skin contacting member 14. In those embodiments in which the length l of the gripping tab 12 is greater than the length L of the skin contacting member 14, the overall length of the device will be defined by the length l, i.e., whichever length is the greatest can define the overall length of the device.

In another embodiment the skin contacting member 14 and gripping tab 12 can be formed of separate pieces that can be the same or different materials. For instance, as illustrated in FIG. 4A, the skin contacting member 14 can be formed with a slot 7. The gripping tab 12 can be sized so as to fit through the slot 7 as shown by the directional arrows and can then be adhered to the skin contacting member 14, for instance at edges 4, 5. Any adherence can be utilized to attach the gripping tab 12 and the skin contacting member 14 firmly to one another, with a preferred adhesive depending upon the materials utilized. For instance, an adhesive bond or a melt bond can be utilized to attach the gripping tab 12 to the skin contacting member 14. Of course, any other method of forming the device can be optionally utilized.

Though generally illustrated as being centered on a skin contacting member 14, it should be understood that a gripping tab 12 can be located at any convenient location on a skin contacting member 14 and in any orientation with respect to the skin contacting member 14. For instance, a gripping tab 12 can be oriented generally in line with a length of the skin contacting member 14 or can be at an angle to the skin contacting member 14 length L. In addition, a gripping tab 12 can be closer to one end and/or side of a skin contacting member 14 than to an opposite end and/or side.

FIG. 4B shows another embodiment in which the gripping tab 12 and the skin contacting member 14 are formed as separate structures and then adhered to one another. In this embodiment, the gripping tab 12 can be adhered to the desired location on the surface of the skin contacting member 14, as shown.

Referring again to FIG. 2, an optional component of an adhesive device can be one or more support films 16, 18. A support film 16. 18 can be utilized to increase the stiffness and resilience of one or both of the gripping tab 12 and the skin contacting member 14.

In one embodiment, the support film 16, 18 may be formed from a material that is transmissive to light, such as transparent or optically diffuse (e.g., translucent) materials. Examples of suitable materials for the support film 16, 18 include, but are not limited to, polymeric materials, such as polystyrene, polypropylene, polyester (e.g., Mylar® film), polybutadiene, polyvinylchloride, polyamide, polycarbonate, epoxides, methacrylates, and polymelamine; and so forth.

The gripping tab 12 and/or the skin contacting member 14 may be cast onto the support film 16, 18, wherein the resulting laminate may be die-cut to the desired size and shape. Alternatively, the gripping tab 12 and/or skin contacting member 14 may simply be laminated to the support 16, 18 with, for example, an adhesive. In some embodiments, a membrane (e.g., nitrocellulose or nylon) is adhered to a Mylar® film. An adhesive is used to bind the membrane to the Mylar® film, such as a pressure-sensitive adhesive.

In one embodiment, the skin contacting member 14 can include perforations, scores, folds, or the like that can be formed over the entire skin contacting member 14 or can be formed in a pattern. When present, such additions can improve and aid the removal of the skin contacting member 14 from the skin surface. For instance, and as described in more detail below, the adhesive device can be used in conjunction with a medical device that is used to insert a needle or other component into a patient. In some embodiments, the inserted device is to be left in the skin, and the ability to easily tear the skin contacting member 14 so as to remove it from around the inserted device can be beneficial. The presence of perforations, scores, etc. across the skin contacting member 14 can aid in such a removal.

In another embodiment, the skin contacting member can include one or more perforations and/or recesses, such as a hole or slot through which a needle or other subdermal device can pass. For example, as illustrated in FIG. 1B, the skin contacting member 14 can include an open slot 20 and/or a hole 22 and/or one or more recesses 24 that can be used for access through the skin contacting member 14 to reach the skin beneath, for instance for placement of a catheter, a needle, an IV line, a dialysis port (e.g., to access a fistula), or the like. For example, a recess 24 can be utilized to access a fistula with an access needle during a dialysis process. Through angling the device while attached to the skin with a recess 24 centered over a fistula, the fistula can be pulled up and open for easier access during dialysis. A slot 20, hole 22, or recess 24 can have any suitable shape and size, a few examples of which are shown in FIG. 1B. In addition, a device can have no such apertures, as illustrated in FIG. 1A, can have a single aperture through the skin contacting member 14, or can have two, three, or more apertures 20, 22, 24 located at any convenient location on the skin contacting member 14, as illustrated in FIG. 1B. In addition, as shown, an aperture can be located at any desired location on the skin contacting member 14.

Though illustrated in FIG. 1 as a generally flat and planar member, the skin contacting member 14 need not necessary be planar. For instance, and as illustrated in FIG. 9-15, the skin contacting member can include surfaces that are contoured and include ridges, bumps, insets, etc. For instance, a skin contacting member can include a convex (i.e., downward curving contour at the bottom surface of the skin contacting member) or concave (i.e., upward curving contour at the bottom surface of the skin contacting member) portion that can better fit the anatomy of a patient. By way of example, when utilized in conjunction with hemodialysis, the fistula is generally just below the skin surface and creates an elevated surface feature on the skin surface, e.g., on the arm. A skin contacting member that includes a concave portion can be formed so as to more closely fit the elevated surface of the fistula area and can lift the skin surface at the fistula so as to improve access of a needle to the fistula. Conversely, a convex skin contacting member surface may be beneficial when accessing a concave surface structure on a patient, for example, above the clavicle or in the anticubital fossa. A skin contacting member can thus be formed so as to mate with the shape of the body at the targeted access point and provide a lifting force to the skin surface for access thereto without compression of the subdermal structures at the access point.

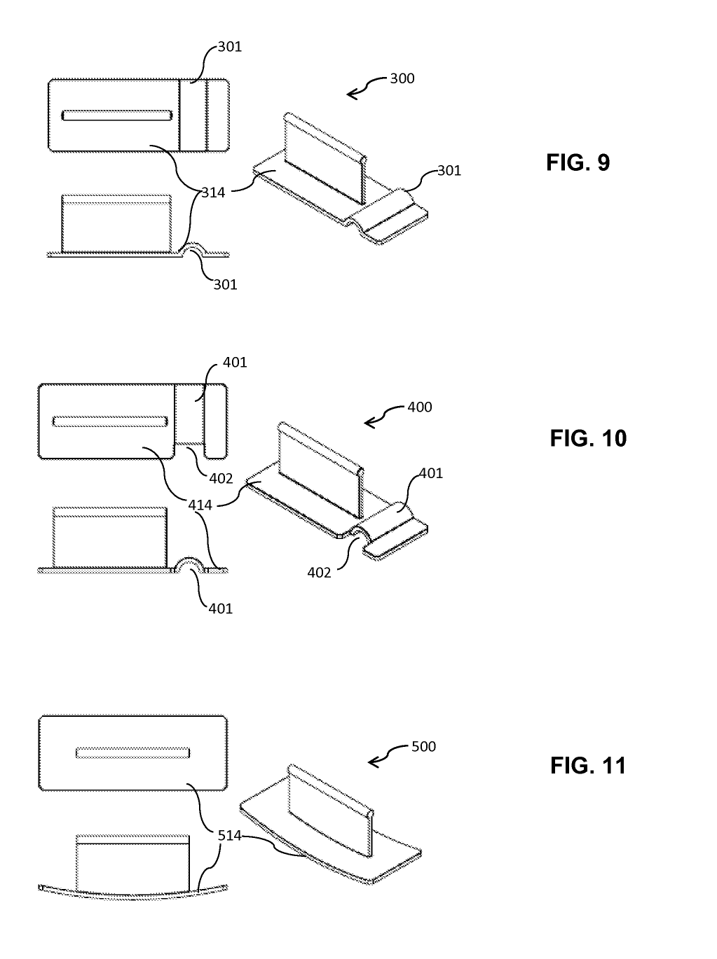

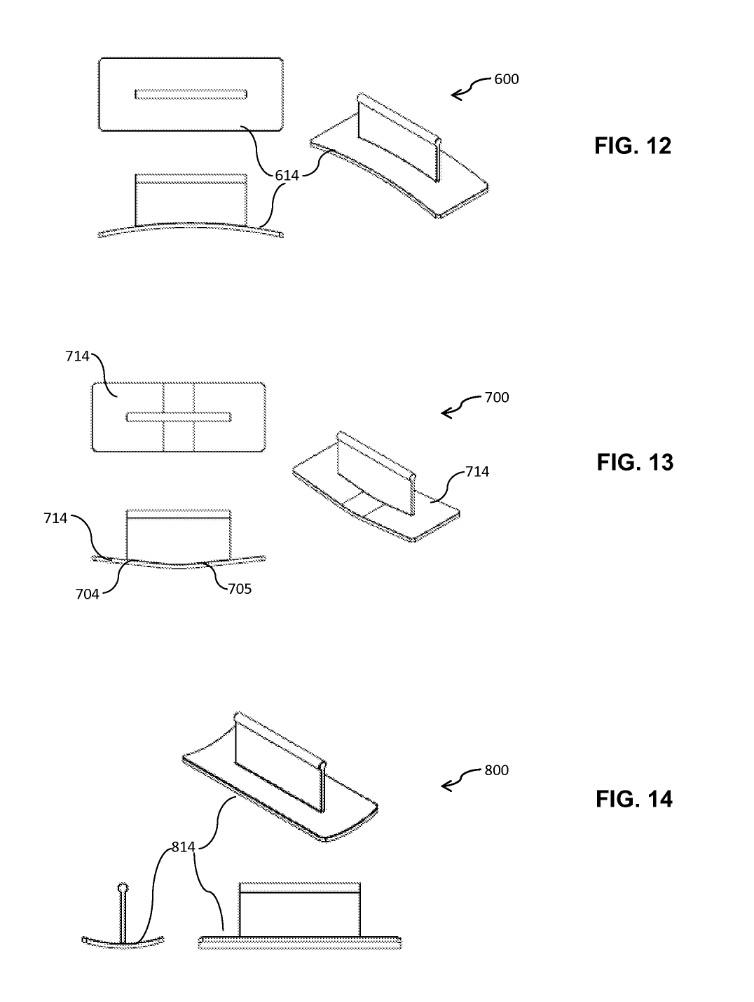

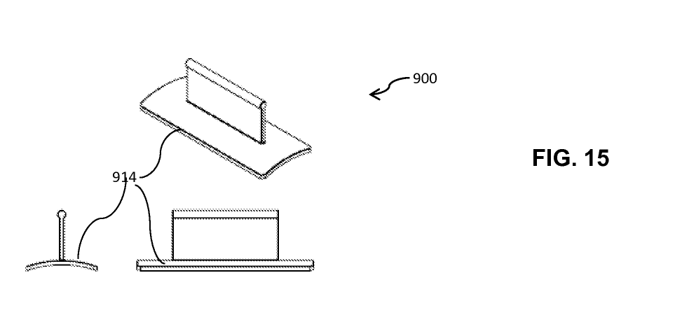

By way of example, and without limitation, FIG. 9 illustrates a skin adhesive device 300 that includes a contoured section 301 that is concave at the lower surface of the device and stretched across the skin contacting member 314 from one side to another. The contoured section 401 of the device 400 of FIG. 10 is similar to that of device 300, but without stretching across the entire width of the skin contacting member 414 thus providing an inset 402 that can be, e.g., utilized for access to a subdermal target. In the device 500 of FIG. 11, the entire skin contacting member 514 has a convex contour, while the skin contacting member 614 of the device 600 of FIG. 12 is concave from end to end, as shown. The device 700 of FIG. 13 also has a generally convex contour, but the convex contour from one end to another of the skin contacting member 714 is formed of a series of two generally flat sections 704, 705 that meet to form an angle between the two surfaces as shown. While the flat sections 704, 705 meet generally along a center line of the device 700 of FIG. 13, this is not a requirement of the device, and portions of a skin contacting member can join at any location of a skin contacting member. In addition, a skin contacting member can be formed of any number of portions that can be joined to form a single member. In addition, contouring of a skin contacting member can be along all or a portion of any width and/or length of a skin contacting member. For instance, in the embodiment of FIG. 14, the device 800 includes a skin contacting member 814 that defines a convex contouring from one side to another across the shorter length of the device, while the device 900 of FIG. 15 includes a skin contacting member 914 that defines a concave contouring from one side to another across the shorter length of the device. In addition, the above are only exemplary embodiments of the skin adhesive device, and any shape for the skin contacting surface is encompassed herein. For instance, while the top view of the skin contacting members of the devices of FIG. 9 through FIG. 15 are generally rectangular, the footprint of the device can vary in any fashion.

The bottom side of the skin contacting member 14 will contact the skin during use, and a biocompatible adhesive is carried on the bottom side of the skin contacting member 14 for adhering the device to the skin. In general, the adhesive can be a pressure sensitive biocompatible adhesive that is suitable for a short-term administration (e.g., for a few minutes to a few hours, generally less than or equal to 1 day) or, in other embodiments, for long-term (e.g., greater than about 1 day, such as for up to about a week, or even longer, such as about 1 week to about 4 weeks). In one aspect, suitable adhesives include, without limitation, acrylic adhesives including cross-linked and uncross-linked acrylic copolymers; vinyl acetate adhesives; natural and synthetic rubbers including polyisobutylenes, neoprenes, polybutadienes, and polyisoprenes; ethylenevinylacetate copolymers; polysiloxanes; polyacrylates; polyurethanes; plasticized weight polyether block amide copolymers, and plasticized styrene-rubber block copolymers or mixtures thereof. In yet another aspect of the invention, contact adhesives for use in the pharmaceutically acceptable carrier layer are acrylic adhesives, such as DuroTak 87-2888 adhesive (National Starch & Chemical Co., Bridgewater, N.J.); and polyisobutylene adhesives such as ARcareJ MA-24 (Adhesives Research, Glen Rock, Pa.) and ethylene vinyl acetate copolymer adhesives. In one embodiment the adhesive can include blends of high and low viscosity polyvinyl ethyl ethers or blends of copolymers of an acrylate ester with acrylic acid. One exemplary adhesive comprises a copolymer, polymerized in acetone, of 47 parts by weight of butyl acrylate, 47 parts of 2-ethylhexyl acrylate and 6 parts of acrylic acid, the copolymer having an intrinsic viscosity of at least 1.9 dl/gm.

The adhesive can be spread continuously or discontinuously over the entire bottom face of the skin contacting member 14, or in a predetermined pattern thereupon. For instance, in those embodiments in which a device such as a needle will be passed through the skin contacting member 14 during the medical procedure, it may be preferred to avoid the presence of the adhesive in that section of the skin contacting member 14 through which the needle will pass. Thus, predetermined areas of the bottom face of the skin contacting member 14 may have little or no adhesive thereon so as to avoid adhesive being picked up and transferred by the needle.

The device 10 also can include a release liner 19 that may be temporarily provided upon the adhesive layer that is applied to the bottom side (side to adhere to the skin) of the skin contacting member 14. The release liner 19 can protect the adhesive layer on the skin contacting member 14 and can be removed prior to attaching the device to the skin. In use, the release liner 19 can be peeled from the adhesive layer just prior to application, and then can be discarded. The release liner 19 can be made of the same materials as the skin contacting member 14, or other suitable materials coated with an appropriate release surface. Thus, while the skin contacting member 14 remains as an integral part of the adhesive device 10 (whether or not the adhesive device 10 is attached to the skin), the release liner 19 will be removed prior to attaching the adhesive device 10 to the skin.

Release liner 19 may be formed of any sheet material such as paper, polyethylene or polypropylene that will adequately protect and be properly released from the adhesive. A suitable release material, for example, is a 40 to 75 pound basis weight paper coated on one or both sides with a suitable finish such as clay and with a release agent such as silicone. In one embodiment, the release liner 19 can include a pull tab 17 or detached edge near an edge of the release liner 19 that may be of the same material and integral with the release liner 19. Alternatively, a pull tab 17 may be of another suitable material and attached to release liner 19 by any suitable manner such as with pressure-sensitive adhesive. Similarly, a pull tab might also conveniently take other forms such as a string or strip attached to the release liner 19. In another embodiment, the release liner 19 can include multiple separate sheet materials, each of which can cover a portion of the bottom side of the skin contacting member 14.

The thickness of release liner 19, skin contacting member 14, gripping tab 12, and any support films 16, 18 can be from about 1 mil to about 30 mils (about 25 micrometers to about 760 micrometers) in some embodiments, or from about 2 mils to about 5 mils (about 50 micrometers to about 125 micrometers) in some embodiments.

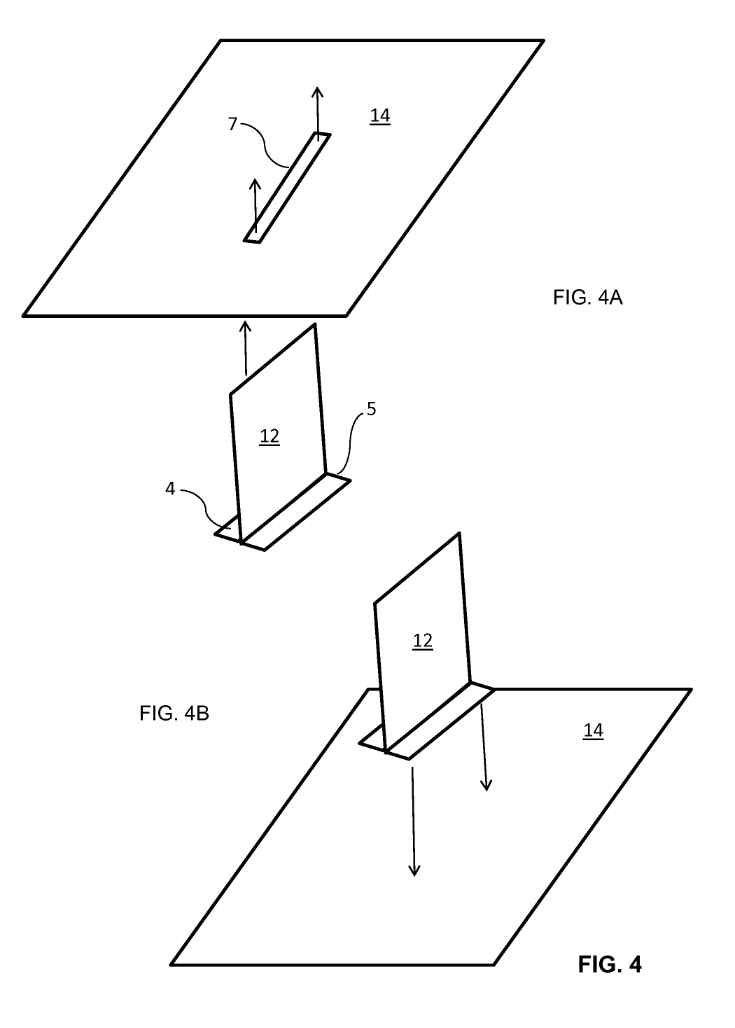

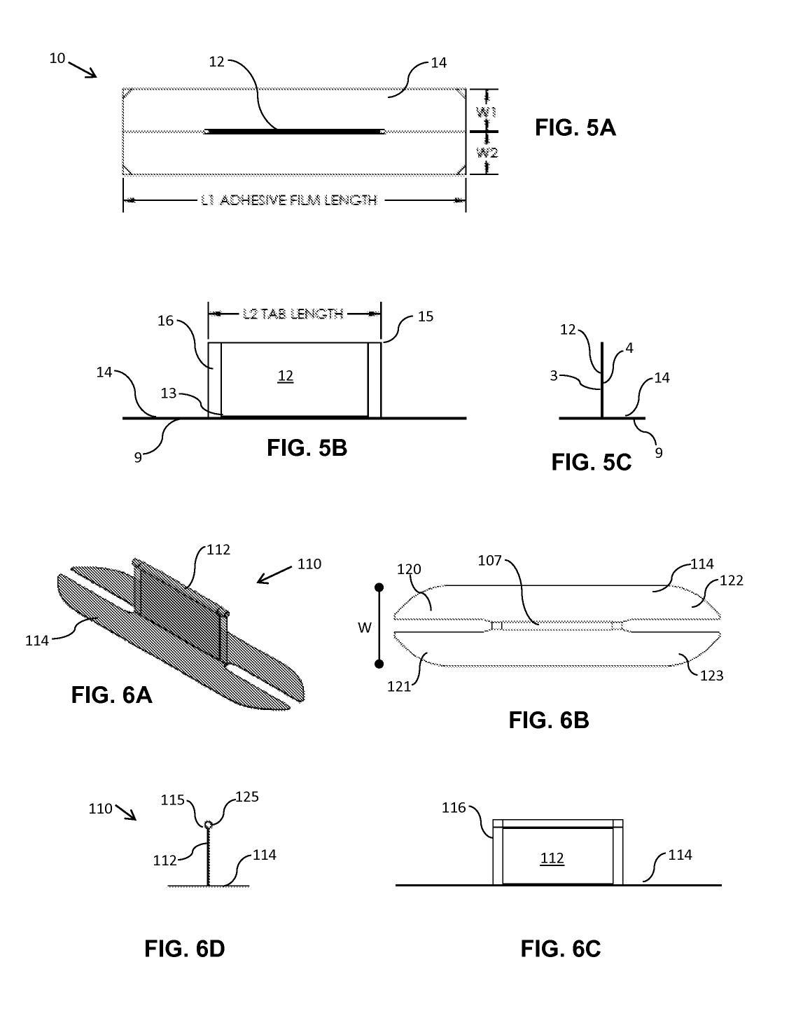

FIG. 5 illustrates a top view (FIG. 5A) a side view (FIG. 5B) and an end view (FIG. 5C) of an adhesive device 10. As can be seen in FIG. 5A, the gripping tab 12 divides the skin contacting member 14 into two widths W1, W2. Though illustrated in this embodiment as approximately equal, this is not a requirement of the device 10 and the widths W1, W2 of the skin contacting member 14 on either side of the gripping tab 12 can differ, though in general there will be at least some width of skin contacting member 14 on either side of the gripping tab 12.

As seen in FIG. 5B, in this embodiment the support film 16 can extend beyond the edges of the inner gripping tab 12. This is not a requirement, however, and in other embodiments, a support film when present can be the same size as the underlying portion gripping tab 12 or even smaller than the underlying portion of gripping tab 12. The height of the gripping tab from the proximal end 13 to the distal end 15 can generally be from about 0.25 inches (about 0.65 centimeter) to about 3 inches (about 7.6 centimeters), or from about 0.5 inches (about 1.3 centimeters) to about 1.5 inches (about 3.8 centimeters) in some embodiments.

To improve the contact of the gripping tab 12 with a medical device, it may prove beneficial in some embodiments to include an adhesive on one side of the gripping tab 12. For instance, either side 3 or side 4 of the gripping tab 12 can include a biocompatible adhesive on all or a portion of the side. The adhesive can be, for instance the same or different as the adhesive applied to the bottom side 9 of the skin contacting member 14. In general, when an adhesive is applied to one side of the gripping tab 12 the device can also include a release liner that covers the adhesive prior to use.

FIG. 6 illustrates another embodiment of a skin adhesive device 110. As can be seen, the device 110 includes a skin contacting member 114, and a gripping tab 112, as described above. A top view of the skin contacting member 114 is shown in FIG. 6B. As can be seen, the skin contacting member 114 includes a slot 107 through which tab 112 can pass and be adheres. Alternatively, gripping tab 112 can be formed as a folded portion of the material that forms skin contacting member 114 or be adhered to the top surface of skin contacting member 114, as discussed previously. The skin contacting member 114 is discontinuous across the width W at either end. This discontinuity across the width W forms separable flaps 120, 121, 122, 123 at the ends of the device 110. These separable flaps 120, 121, 122, 123 can be used to better position the device 110 on a particular location on the body, for instance on a small area such as the wrist. Such variations in geometry can be used to shape a device for any particular location on the body. For instance, separable flaps can be formed on only a single end of a skin contacting member, or multiple flaps can be formed on one or more ends and/or off of the side of the device. In another embodiment, the flaps can include a slit within the flaps, with the end of each flap being contiguous. There is no particular shape required for the skin contacting member 114 of the device 110.

FIG. 6D presents an end view of the device 110. The device 110 includes a rod 125 at the distal end 115 of the gripping tab 112. Such a formation can be utilized when holding the gripping tab 112 against a surface of a medical device. For instance, the medical device to be used in conjunction with the device 110 can include a negative of the formation (e.g., an inset that mates with the rod 125) to mate with the formation so as to properly align and hold the gripping tab 112 against the surface of the medical device. Any shape for the formation can be used such as a rod, a spherical, hemispherical, rectangular, star-shaped, or any other shaped formation, as well as a series of formations, e.g., raised dots or ridges, in a geometric or random pattern. In addition, though illustrated at the distal end 115 of the gripping tab 112, a formation such as a rod 125, when present, can be located at any convenient place on the gripping tab 112.

As can be seen in FIG. 6C, this embodiment of a device also includes a support film 116 as a component of the gripping tab 112 that extends beyond the edges of the underlying portion of the gripping tab 112.

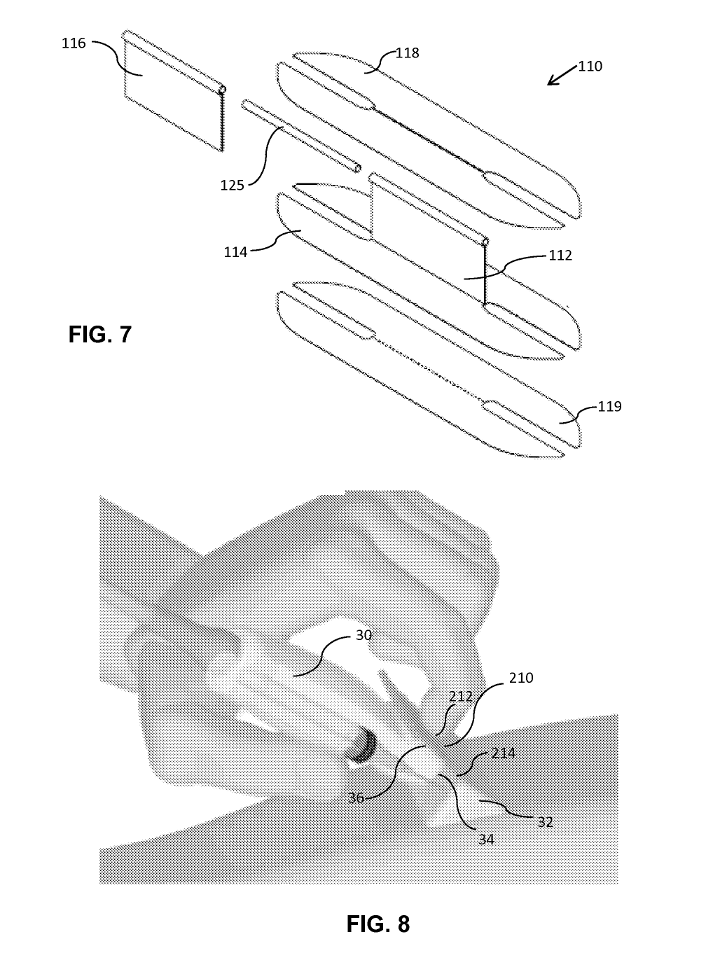

FIG. 7 provides an exploded view of the device 110 including gripping tab 112, skin contacting member 114, and release liner 119. The device 110 also includes support films 116, 118 as described previously and a rod 125 for mating with a negative similar formation on a medical device, as described.

In one embodiment, a skin adhesive device can be utilized in conjunction with any of a variety of medical devices for improvement of the contact between the medical device and the skin. By way of example, FIG. 8 illustrates one embodiment of the use of a skin adhesive device 210 with a medical device 30. Medical device 30 can be, e.g., an ultrasound transducer, an insertion cartridge for inserting a subdermal device such as a needle or a biopsy device, a removal cartridge for removing fluids, solids, implanted devices, etc. from under the skin of a subject, or any other medical device that is held against the skin surface during a procedure.

The medical device 30 can include a surface 34 that is held against the skin 32 during the procedure. During use, the skin contacting member 214 of the adhesive device 210 is adhered to the surface of the skin 32 by use of the biocompatible adhesive that is on the bottom surface of the skin contacting member 214. The gripping tab 212 can then be gripped against a surface 36 of the medical device. As described, this grip can be aided by the inclusion of an adhesive on one side of the gripping tab 212 and/or the inclusion of a formation on the gripping tab 212 and mated insertion on the surface 36 of the medical device 30.

When the gripping tab 212 is held against a surface 36 of the device 30, another surface 34 of the device 30 is held against the skin contacting member 214 and the skin 32. As the device 30 is moved during the procedure, e.g., during visualization, insertion, removal, etc., the skin 32 will remain held adjacent to the surface 34 by the adhered skin contacting member 214. This will thus prevent relative motion between the skin 32 at that area that is adjacent to the surface 34 of the device 30.

The device also can pull the skin upward during a procedure, which can prevent compression of subdermal features during a procedure. This can be particularly beneficial during procedures that involve the puncture or cutting of the skin. The upward force on the skin provided by the disclosed device can oppose the puncture or cutting force of a medical device utilized to access subdermal formations and as the subdermal features will be less compressed, targeting of the subdermal device (e.g., a needle, trocar, biopsy device, scalpel, dialysis device, etc.) to the subdermal features can be improved. For instance, use of the disclosed device can prevent excessive compression of a subdermal target by a medical device (e.g., an ultrasound) as the subdermal target of the procedure is not excessively compressed during access by the puncture/cutting force. Thus, in some embodiments, the skin adhesive device can be utilized in conjunction with a medical device such as a needle or biopsy device that is not necessarily held in conjunction with the skin adhesive device during use. For instance, a care giver can utilize the device to pull the skin taut and prevent compression of underlying structures, such as a vein or a dialysis fistula, and the underlying structure can be accessed through or near the device by use of a subdermal probe, needle, etc.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

1. A skin adhesive device comprising:
a gripping tab, the gripping tab including a proximal end and a distal end and a first side and a second side;
a skin contacting member, the skin contacting member including a top surface and a bottom surface and having a length and a width, the gripping tab extending from the top surface of the skin contacting member with the proximal end of the gripping tab nearer the top surface of the skin contacting member as compared to the distal end of the gripping tab, the bottom surface of the skin contacting member carrying a biocompatible adhesive; and

2. The skin adhesive device of claim 1, further comprising a release liner that overlays the biocompatible adhesive.

3. The skin adhesive device of claim 1, wherein the gripping tab and the skin contacting member are formed of the same material.

4. The skin adhesive device of claim 1, wherein the gripping tab includes a biocompatible adhesive on the first side.

5. The skin adhesive device of claim 1, the gripping tab and/or the skin contacting member including a support film.

6. The skin adhesive device of claim 1, the gripping tab further comprising a formation that increases a width of the gripping tab.

7. The skin adhesive device of claim 1, wherein the gripping tab or the skin contacting member comprise a polymer and/or a natural or synthetic fiber.

8. The skin adhesive device of claim 1, wherein the skin contacting member comprises perforations, scores, two or more separable flaps, a channel, a hole or a recess in the skin contacting member.

9. The skin adhesive device of claim 1, wherein the gripping tab divides the skin contacting member into a first width and a second width that are approximately equal to one another.

10. The skin adhesive device of claim 1, wherein the skin contacting member is discontinuous across the width of the skin contacting member and the discontinuity forms an aperture through which the proximal end of the gripping tab is held.

11. The skin adhesive device of claim 1, wherein the biocompatible adhesive covers the entire bottom surface of the skin contacting member.

12. A method for holding skin taut during a medical procedure, the method comprising:
adhering the biocompatible adhesive on the bottom of the skin contacting member of the skin adhesive device of claim 1 to the skin; and
maintaining a puffing pressure on the gripping tab during the medical procedure, the pulling pressure pulling the skin adhered to the biocompatible adhesive and maintaining the skin taut.

13. The method of claim 12, further comprising:
placing a first surface of a medical device adjacent to at least a portion of the top surface of the skin contacting member;
placing a second surface of the medical device adjacent to the first side of the gripping tab;
holding the gripping tab against the medical device as the medical device is used to carry out a medical procedure.

14. The method of claim 13, wherein the medical device is an ultrasound device.

15. The method of claim 13, wherein the medical procedure comprises tipping the medical device and stretching the skin that is adhered to the bottom surface of the skin contacting member.

16. The method of claim 12, further comprising removing a release liner from the skin adhesive device.

17. The method of claim 12, wherein the medical procedure comprises inserting a subdermal device into the skin.

18. The method of claim 12, wherein the medical procedure comprises visualizing a subdermal area.

19. The skin adhesive device of claim 3, wherein the gripping tab and the skin contacting member are formed of a single piece of the material.

20. The skin adhesive device of claim 1, wherein the gripping tab divides the skin contacting member is a first width and second width, the first and second width being unequal to one another.

21. The skin adhesive device of claim 1, wherein the biocompatible adhesive covers only a portion of the bottom surface of the skin contacting member.
